SEQUENCE LISTING (12 sequences)

SEQ ID NO: 1 - protein, 1122 aa, Artificial Sequence
  Description of Artificial Sequence Synthetic polypeptide
MLAKGLPPRSVLVKGCQTFLSAPREGLGRLRVPTGEGAGISTRSPRPFNEIPSPGDNGWLNLYHFWRETGTHKVHLHHVQNFQKYGPIYREKLGNVESVYVIDPEDVALLFKSEGPNPERFLIPPWVAYHQYYQRPIGVLLKKSAAWKKDRVALNQEVMAPEATKNFLPLLDAVSRDFVSVLHRRIKKAGSGNYSGDISDDLFRFAFESITNVIFGERQGMLEEVVNPEAQRFIDAIYQMFHTSVPMLNLPPDLFRLFRTKTWKDHVAAWDVIFSKADIYTQNFYWELRQKGSVHHDYRGILYRLLGDSKMSFEDIKANVTEMLAGGVDTTSMTLQWHLYEMARNLKVQDMLRAEVLAARHQAQGDMATMLQLVPLLKASIKETLRLHPISVTLQRYLVNDLVLRDYMIPAKTLVQVAIYALGREPTFFFDPENFDPTRWLSKDKNITYFRNLGFGWGVRQCLGRRIAELEMTIFLINMLENFRVEIQHLSDVGTTFNLILMPEKPISFTFWPFNQEATQQTDGTSSTQEKTPQICVVGSGPAGFYTAQHLLKHPQAHVDIYEKQPVPFGLVRFGVAPDHPEVKNVINTFTQTAHSGRCAFWGNVEVGRDVTVPELREAYHAVVLSYGAEDHRALEIPGEELPGVCSARAFVGWYNGLPENQELEPDLSCDTAVILGQGNVALDVARILLTPPEHLERTDITKAALGVLRQSRVKTVWLVGRRGPLQVAFTIKELREMIQLPGARPILDPVDFLGLQDKIKEVPRPRKRLTELLLRTATEKPGPAEAARQASASRAWGLRFFRSPQQVLPSPDGRRAAGVRLAVTRLEGVDEATRAVPTGDMEDLPCGLVLSSIGYKSRPVDPSVPFDSKLGVIPNVEGRVMDVPGLYCSGWVKRGPTGVIATTMTDSFLTGQMLLQDLKAGLLPSGPRPGYAAIQALLSSRGVRPVSFSDWEKLDAEEVARGQGTGKPREKLVDPQEMLRLLGHTDGASSSSEDKITVHFINRDGETLTTKGKVGDSLLDVVVENNLDIDGFGACEGTLACSTCHLIFEDHIYEKLDAITDEENDMLDLAYGLTDRSRLGCQICLTKSMDNMTVRVPETVADARQSIDVGKTSDYKDDDDK

SEQ ID NO: 2 - protein, 427 aa, Unknown
  Description of Unknown Cholesterol dehydrogenase sequence
MPTLYKKAGFQLGSLENLYFQGHHHHHHGTMAGFLNCCPGCCMEPGASDYKDDDDKMLRRMGDASLTTELGRVLVTGGAGFVGANLVTTLLDRGHWVRSFDRAPSLLPAHPQLEVLQGDITDADVCAAAVDGIDTIFHTAAIIELMGGASVTDEYRQRSFAVNVGGTENLLHAGQRAGVQRFVYTSSNSVVMGGQNIAGGDETLPYTDRFNDLYTETKVVAERFVLAQNGVDGMLTCAIRPSGIWGNGDQTMFRKLFESVLKGHVKVLVGRKSARLDNSYVHNLIHGFILAAAHLVPDGTAPGQAYFINDAEPINMFEFARPVLEACGQRWPKMRISGPAVRWVMTGWQRLHFRFGFPAPLLEPLAVERLYLDNYFSIAKARRDLGYEPLFTTQQALTECLPYYVSLFEQMKNEARAEKTAATVKPG

SEQ ID NO: 3 - protein, 510 aa, Unknown
  Description of Unknown 3-ketosteroid delta1-dehydrogenase sequence
MQDWTSECDVLVVGSGGGALTGAYTAAAQGLTTIVLEKTDRFGGTSAYSGASIWLPGTQVQERAGLPDSTENARTYLRALLGDAESERQDAYVETAPAVVALLEQNPNIEFEFRAFPDYYKAEGRMDTGRSINPLDLDPADIGDLAGKVRPELDQDRTGQDHAPGPMIGGRALIGRLLAAVQSTGKAELRTESVLTSLIVEDGRVVGAEVESGGETQRIKANRGVLMAAGGIEGNAEMREQAGTPGKAIWSMGPFGANTGDAISAGIAVGGATALLDQAWFCPGVEQPDGSAAFMVGVRGGLVVDSAGERYLNESLPYDQFGRAMDAHDDNGSAVPSFMIFDSREGGGLPAICIPNTAPAKHLEAGTWVGADTLEELAAKTGLPADALRSTVEKFNDAAKLGVDEEFHRGEDPYDAFFCPPNGGANAALTAIENGPFYAARIVLSDLGTKGGLVTDVNGRVLRADGSAIDGLYAAGNTSASLSGRFYPGPGVPLGTAMVFSYRAAQDMAK

SEQ ID NO: 4 - protein, 595 aa, Unknown
  Description of Unknown Anoxic cholesterol metabolism beta enzyme sequence
MPTLYKKAGFQLGSGTENLYFQGTMSIETNTYDVIVVGSGAGAMLAAARAHDLGLSVLVVEKSDKYGGTSAVSGGAVWIPNNSQMQIKDSFDEALTYLKAATQGLVAEDRLLAYLESAPQMVEYINANMTLQYFPCHRYPDYYQHLPGAKPGGRTMEPMLFDAALLGDEFANLRMAYTGTLLMGKASMTATEAHVMLAKEPGWMLQVIKSLGRYYLDLPWRLKSRHDRKRGLGNAMAAGLRHALLERKVPLWLNTPFESLITEGAENKRVTGIVVKRNGQTLQLTARRGVVLGAGGFERNQQMREQYLPKPTNAAWSATPPHNTGDTIRAAMDIGARAELMDWAWWVPSIHVPGEAAQTGLFAERNLPGCIVVNGKGQRFINEASPYLEFGAAMYENHARSGSAVPAWLIFDGKFRYNYPMGPLMPGQIQPDRKAWLGKVYWRDDTLEGLAKQIGVDAAGLKQSVELNNQYAQDGKDREFDKGGNVFDRYYGDYNVKPNPCLAPIGKPPYYAMRVDAGDIGTKGGLLTDKDARVLDESDRPIEGLYCIGNNSASVMGKAYPGAGGTLGPAMTFGFRAANHIAASKYPYDVPDYAG

SEQ ID NO: 5 - protein, 860 aa, Unknown
  Description of Unknown 3-ketosteroid 9alpha-hydroxylase sequence
MAPYSLLVTRLQKALGVRQYHVASVLCQRAKVAMSDYKDDDDKGGGGSMSIDTARSGSDDDVEIREIQAAAAPTRFARGWHCLGLLRDFQDGKPHSIEAFGTKLVVFADSKGQLNVLDAYCRHMGGDLSRGEVKGDSIACPFHDWRWNGKGKCTDIPYARRVPPIAKTRAWTTLERNGQLYVWNDPQGNPPPEDVTIPEIAGYGTDEWTDWSWKSLRIKGSHCREIVDNVVDMAHFFYIHYSFPRYFKNVFEGHTATQYMHSTGREDVISGTNYDDPNAELRSEATYFGPSYMIDWLESDANGQTIETILINCHYPVSNNEFVLQYGAIVKKLPGVSDEIAAGMAEQFAEGVQLGFEQDVEIWKNKAPIDNPLLSEEDGPVYQLRRWYQQFYVDVEDITEDMTKRFEFEIDTTRAVASWQKEVAENLAKQAEGSTATPGSGATNFSLLKQAGDVEENPGPMAPYSLLVTRLQKALGVRQYHVASVLCQRAKVAMSGYPYDVPDYAGGGGSMTAVQAPVTSRATVLTVSAVVQETADAVSLVFDVPDDRREDFTYRPGQFLTLRIPSDRTGSVARCYSLASSPFTGEPPKVTVKRTAGGYGSNWLCDNIVAGRSIEVLPPAGVFTPADLTEKLVLFAGGSGITPVMSILESALHSGNRDVVLIYGNRDEKSVIFAEKLRELAARHAGALTVVHWLESVQGLPSPQQLATLISPFADHRAYMCGPGPFMDTVREGLLLAGVPKDRIHAEVFTSLSGDPFADVPLVEIDESDADATSATVQLDGEEHDLVWPRSATLVDVMLSKGLDVPYSCREGECGSCACTVVEGDVDSLPSAILDEEDIANGYVLACQARPKSDHVRIEF

SEQ ID NO: 6 - protein, 372 aa, Unknown
  Description of Unknown 3beta-hydroxysteroid dehydrogenase sequence
MGWSCLVTGAGGLLGQRIVRLLVEEKELKEIRALDKAFRPELREEFSKLQNRTKLTVLEGDILDEPFLKRACQDVSVVIHTACIIDVFGVTHRESIMNVNVKGTQLLLEACVQASVPVFIYTSSIEVAGPNSYKEIIQNGHEEEPLENTWPTPYPYSKKLAEKAVLAANGWNLKNGDTLYTCALRPTYIYGEGGPFLSASINEALNNNGILSSVGKFSTVNPVYVGNVAWAHILALRALRDPKKAPSVRGQFYYISDDTPHQSYDNLNYILSKEFGLRLDSRWSLPLTLMYWIGFLLEVVSFLLSPIYSYQPPFNRHTVTLSNSVFTFSYKKAQRDLAYKPLYSWEEAKQKTVEWVGSLVDRHKETLKSKTQ

SEQ ID NO: 7 - protein, 285 aa, Homo sapiens
MLLATFKLCAGSSYRHMRNMKGLRQQAVMAISQELNRRALGGPTPSTWINQVRRRSSLLGSRLEETLYSDQELAYLQQGEEAMQKALGILSNQEGWKKESQQDNGDKVMSKVVPDVGKVFRLEVVVDQPMERLYEELVERMEAMGEWNPNVKEIKVLQKIGKDTFITHELAAEAAGNLVGPRDFVSVRCAKRRGSTCVLAGMATDFGNMPEQKGVIRAEHGPTCMVLHPLAGSPSKTKLTWLLSIDLKGWLPKSIINQVLSQTQVDFANHLRKRLESHPASEARC

SEQ ID NO: 8 - protein, 145 aa, Homo sapiens
MVGRNSAIAAGVCGALFIGYCIYFDRKRRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAEDDVE

SEQ ID NO: 9 - protein, 375 aa, Artificial Sequence
  Description of Artificial Sequence Synthetic polypeptide
MVGRNSAIAAGVCGALFIGYCIYFDRKRRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAEDDVEGSDDDDKLEETLYSDQELAYLQQGEEAMQKALGILSNQEGWKKESQQDNGDKVMSKVVPDVGKVFRLEVVVDQPMERLYEELVERMEAMGEWNPNVKEIKVLQKIGKDTFITHELAAEAAGNLVGPRDFVSVRCAKRRGSTCVLAGMATDFGNMPEQKGVIRAEHGPTCMVLHPLAGSPSKTKLTWLLSIDLKGWLPKSIINQVLSQTQVDFANHLRKRLESHPASEARC

SEQ ID NO: 10 - protein, 418 aa, Artificial Sequence
  Description of Artificial Sequence Synthetic polypeptide
MLLATFKLCAGSSYRHMRNMKGLRQQAVMAISQELNRRALGGPTPSTWINQVRRRSSLLGSRLEETLYSDQELAYLQQGEEAMQKALGILSNQEGWKKESQQDNGDKVMSKVVPDVGKVFRLEVVVDQPMERLYEELVERMEAMGEWNPNVKEIKVLQKIGKDTFITHELAAEAAGNLVGPRDFVSVRGSDDDDKLEETLYSDQELAYLQQGEEAMQKALGILSNQEGWKKESQQDNGDKVMSKVVPDVGKVFRLEVVVDQPMERLYEELVERMEAMGEWNPNVKEIKVLQKIGKDTFITHELAAEAAGNLVGPRDFVSVRCAKRRGSTCVLAGMATDFGNMPEQKGVIRAEHGPTCMVLHPLAGSPSKTKLTWLLSIDLKGWLPKSIINQVLSQTQVDFANHLRKRLESHPASEARC

SEQ ID NO: 11 - DNA, 2565 bp, Homo sapiens
gcagctgcgggactcagaggcgaagcttgaggggctcaggaaggacgaagaaccacccttgagagaagaggcagcagcagcggcggcagcagcagcggcagcgaccccaccactgccacatttgccaggaaacaatgctgctagcgacattcaagctgtgcgctgggagctcctacagacacatgcgcaacatgaaggggctgaggcaacaggctgtgatggccatcagccaggagctgaaccggagggccctggggggccccacccctagcacgtggattaaccaggttcggcggcggagctctctactcggttctcggctggaagagactctctacagtgaccaggagctggcctatctccagcagggggaggaggccatgcagaaggccttgggcatccttagcaaccaagagggctggaagaaggagagtcagcaggacaatggggacaaagtgatgagtaaagtggtcccagatgtgggcaaggtgttccggctggaggtcgtggtggaccagcccatggagaggctctatgaagagctcgtggagcgcatggaagcaatgggggagtggaaccccaatgtcaaggagatcaaggtcctgcagaagatcggaaaagatacattcattactcacgagctggctgccgaggcagcaggaaacctggtggggccccgtgactttgtgagcgtgcgctgtgccaagcgccgaggctccacctgtgtgctggctggcatggccacagacttcgggaacatgcctgagcagaagggtgtcatcagggcggagcacggtcccacttgcatggtgcttcacccgttggctggaagtccctctaagaccaaacttacgtggctactcagcatcgacctcaaggggtggctgcccaagagcatcatcaaccaggtcctgtcccagacccaggtggattttgccaaccacctgcgcaagcgcctggagtcccaccctgcctctgaagccaggtgttgaagaccagcctgctgttcccaactgtgcccagctgcactggtacacacgctcatcaggagaatccctactggaagcctgcaagtctaagatctccatctggtgacagtgggatgggtggggttcgtgtttagagtatgacactaggattcagattggtgaaagtttttagtaccaagaaaacagggatgaggctcttggattaaaaggtaacttcattcactgattagctatgacatgagggttcaggcccctaaaataattgtaaaactttttttctgggcccttatgtacccacctaaaaccatctttaaaatgctagtggctgatatgggtgtgggggatgctaaccacagggcctgagaagtcttgctttatgggctcaagaatgccatgcgctggcagtacatgtgcacaaagcagaatctcagagggtctcctgcagccctctgctcctcccggccgctgcacagcaacaccacagaacaagcagcaccccacagtgggtgccttccagaaatatagtccaagctttctctgtggaaaaagacaaaactcattagtagacatgtttccctattgctttcataggcaccagtcagaataaagaatcataattcacacaaacatcagtctttgttttaatattgtactgttaaaaaaatctatgcagctgggtgcagtggctcacgcctgtaatcccagcattttgggaggctgaggtaggcggatcacaaggtcaggagatcgagaccatcctggccaacatggtgaaaccccgtctctactaaaatacaaaaaattagctgggtgtggtggcgcaaacctgtagtcgtagctacttgggaggctgaggcaggggaatcacttgaaccccggaggcggaggttgtagtgggccgagattgtgccactgcgctccagcgtgggcgacagagtgagactccatctcaaaaaaaaaaaaaaaaaaatctatgctagtagattacaacttcacactagaggagttctggacaaagcttttaattagtcaaactaaattaaggctcattaaaaggaaaggaactactgggaaattatgcaattcaataatttagactctgttaccaggatctttcataaaaatttaatttccataatcataacctaaatgagttcttaaagaattctataagcaatagctgattaatgggccctggaagatgaagattataactgtttatttacctaattaaaaggaaaggcagtgccaaatatgagaggataaacaatattagttaacatttctgttatttatgatgccaattagtagtaagataattccacagctgtcaactttgtttggggctggcaacttctctgcttaaacaggctaaaagtttagtattctgggagaagtggctggaagaaggggtaatatggtgaaagcaaattccctttcccaggagtcaagagaatttatgtgaggtggctcccgcctgtaatctcagcactttgggaggccaaggcaggcaatcacctgagggcaggagaccagcctgactaacatggagaaaccccatctctactaaaaatac

SEQ ID NO: 12 - DNA, 3283 bp, Homo sapiens
ctttctgtgttcctggcccgcggccgtcgggtgtgagctgcgccgaccgctctgagggttcgtggcccaccgctccttcgcggtccctgccgccaccgtccacgctcagcgttgtagagaagatggtgggtcggaacagcgccatcgccgccggtgtatgcggggcccttttcattgggtactgcatctacttcgaccgcaaaagacgaagtgaccccaacttcaagaacaggcttcgagaacgaagaaagaaacagaagcttgccaaggagagagctgggctttccaagttacctgaccttaaagatgctgaagctgttcagaagttcttccttgaagaaatacagcttggtgaagagttactagctcaaggtgaatatgagaagggcgtagaccatctgacaaatgcaattgctgtgtgtggacagccacagcagttactgcaggtcttacagcaaactcttccaccaccagtgttccagatgcttctgactaagctcccaacaattagtcagagaattgtaagtgctcagagcttggctgaagatgatgtggaatgagaaacaaatgtcaacataataaaatctcagttaaaaatattttaaaaattcttggtagttgagcagctctgggggaataagggcaaatatgcttgttatgaactacactgaaatctaccaaagttaatgtttactttgtgtagatccatttgtctattttatttatttttcccagtgaaaagtgtattttgatagagaacttttcattctataaatacactatgagttactaaaatatcatggattttgtttattcctgaaacatagttacatagttaaactgtacatatgacatggcttatgttaaaaatacccagtgctcagttttgaaagataggcaaaaaaaaaaaagtataggagaaactgaagaatgtacacttttttagagggcacattttgctgtaaatctggaaatttgatagacttgactgtgtttgtgaaaactgagcattaaaggttttgattgatcctttctttccatttaatctctgagacgtaaatatgtgaggtgtgctgctgtgctgggttaacagcttccttccctttctgtgtagcagtcttgaaatgttctgtttaaatcagtaggcttaatgtgttctgggtatttatctccttgtattttaaatatatgtagttgcaaatagcaccaggaattagatttctgtacacccctaatctagccttgtgagcttcgctagttaatgtgtgctcactttccctccatttgttacgtgagagaatgcgtctgctgatcactgaagtgtcccttttagcttctgattcattgggttctgttgggcatctttaaatccaccttaacctgaggaatgtatgtgggcaaccaggccctgcatttttttatattctgaattttgcatgcttgcctgacttagtatttctgaattgatgttttttttaatggtataactatcttgattttcactgaaattatatggttctgtcactactctgtaaattaatccgaaacttttaaggtaactgggatgatctgcttgtaaaaatgcttgttgccttttgctttatcttcagtgtacctccttaatcctgcttcaacttgattatcttgtgaaacgatgagagtaagttgcaaccttgtgactgaaaacttgaaaagagtggagcaggtgggacctcttattctcaaatagtgacatattctccgtagtcacagtttcagaactgagtaaggatccttggtacttggtggcatctgttgaactgaggagcatttctcattgtaaagattgcctttgttctgtctaaaagtctggagaaatcccaaagacttttcctatgtactaggcattttattttgattgacttacaaactcttcttaatcattatcaatctcggtttttttgtggtgcagtggaaggagaaataggtctagtttctgcctctgattagccgcacagccttgaacaaatcacatttcatctttgaacttacctctactgttagactaggcgactcacatttgaggacttttctcgggtatcttgagggtttgtgatcctgaacccttaaacagtgcttttttgttacacaggagggcttttttggggggatgaccagtacagacatgccagttagttttactagtgggatcccaaatccaaagcagtgtagtggtgattggtcagtgactaaccaggcagctaagaagtcttaggcagcagcccagacatgtatagaggggcagttagagggagaacaggggtgggaaagggagcaaggggcagatagctcagcaaggaaagaatgggctcagaaaaaggagggctggctggaggagtgaggggcagcttaagtttggggagggtagaaacgccgtttccttgggaactggagtgcagtatgagctgggtgtcacttggctctgaacatactggctttgctgtaatgcttgaaaaggcgttggtatcttcattttacagttcattaacccaagtacgttttcttatttaaatgacaactttggtgctttaaaatgaggtaccactttttaaagctagctgtgtcgagttaaagaaaaaatcagcagttttttctcccagaaatgtaattgccaaacactattcatccccatcttaagttttacaaggtgatgtaatcagcttgttgtagtgatgctggccaaatggtgctcagcaggtgagaacaaaaaaaccccagatctcagtgaactaatacacagcttgagcgtttccatgtgctaatgttgcacacttactaaaaaactttggaaatggaaaataatgtattagtgcaacagttgatgtgcttctttgggcaaagatatagttttgttccacaatttgtacttaaaagcgaaagaacattgaaaacatagacttactggctgtagcaatgctggcctgttaactgataactagaacttaggttcacgtttatgtaaagtgtgtaaaacctagtagagcttgcatagtcggcactcagtaaatgtttggttccttttgccccttggtaagtttattttaccatcctcccacctgccattctgactttattaaatcaacatgtggaccagagtgttaatgagatgttattgcagaagagattgagaaaattggtatatcatgcagataacatacaaaatctttttgtaacgtaaaaaatgcagttttattattgcttgtgcctcaactgtttaagtgaatattaaagggcttggagaaaa

(19) United States
(12) Patent Application Publication
(10) Pub. No.: US 2024/0050592 A1
(43) Pub. Date: Feb. 15, 2024

(54) ENHANCING MITOCHONDRIAL-BASED FLOW AND CATABOLISM OF CHOLESTEROL

(71) Applicant: REPAIR BIOTECHNOLOGIES, INC., Syracuse, NY (US)

(72) Inventors: Mourad Topors, Scarborough (CA); Reason, Syracuse, NY (US); Guilherme Cherman Perdigao de Oliveira, Rio de Janeiro (BR); Marc Ridilla, Camillus, NY (US); David Mackenzie-Liu, Syracuse, NY (US); Garrett Strough, Lorraine, NY (US)

(21) Appl. No.: 18/265,376

(22) PCT Filed: Dec. 8, 2021
(86) PCT No.: PCT/US2021/062389

§ 371 Date: Jun. 5, 2023

Related U.S. Application Data
(60) Provisional application No. 63/122,886, filed on Dec. 8, 2020.

(51) Int. Cl. A61K 48/00; C07K 14/415; C07K 14/705; C07K 14/47; A61P 3/06; A61K 38/17; A61K 38/44; C12N 15/85; C12N 9/02; C12N 5/071

(52) U.S. Cl. CPC A61K 48/0058; C07K 14/415; C07K 14/705; C07K 14/4705; A61P 3/06; A61K 38/1709; A61K 38/443; C12N 15/85; C12N 9/0081; C12Y 114/15006; C12N 5/0686; C07K 2319/50; C12N 2800/107

(57) ABSTRACT

Disclosed herein are compositions, methods, and systems for enhancing cholesterol degradation in a cell, tissue, or organism. In many embodiments, the disclosure describes the use of one or more proteins, or sequences coding therefor, to enhance flow of cholesterol into the mitochondrion, where the cholesterol is degraded by one or more proteins comprising bacteria-related sequences. The compositions, methods, and systems disclosed herein are useful in the prevention or treatment of diseases, disorders, and conditions associated with high levels of cholesterol in the blood or cells of a patient.

### CROSS-REFERENCE TO RELATED APPLICATIONS

This application claims benefit of priority pursuant to 35 U.S.C. § 119(e) of U.S. provisional patent application No. 63/122,886 entitled “Enhancing mitochondrial-based flow and catabolism of cholesterol,” filed on 8 Dec. 2020, which is hereby incorporated by reference in its entirety.

### FIELD

The disclosed processes, methods, and systems are directed to treating a subject with high levels of cholesterol or at risk thereof by enhancing cholesterol shuttling into and degradation within the mitochondria.

### SEQUENCE LISTING

The instant application contains a Sequence Listing which has been submitted electronically in ASCII format and is hereby incorporated by reference in its entirety. Said ASCII copy, created on Dec. 7, 2021, is named P289347_WO_01_SL.txt and is 52,905 bytes in size.

### BACKGROUND

High cholesterol (or hypercholesterolemia) is the presence of high levels of cholesterol in the blood that may be the result of poor diet, lifestyle, disease (e.g. diabetes) or genetics. High concentrations of cholesterol within the cell is a characteristic of several diseases, for example fatty liver disease, atherosclerosis, etc.

Atherosclerosis involves narrowing of arteries resulting from a buildup of cholesterol-laden lipoproteins within the arterial wall. Often a precursor to leading causes of death including cardiovascular disease (CVD), myocardial infarction, stroke and peripheral vascular disease. Many current therapies for these diseases focus on preventing a second event (or a primary event in high-risk individuals) by reducing the circulating levels of low density lipoproteins (LDLs) and/or increasing high-density lipoproteins (HDLs), because although mammalian cells are able to synthesize cholesterol, they cannot break it down (catabolize it).

What is needed are compositions and methods for reducing lipid and cholesterol levels in subjects suffering from excess lipid and cholesterol.

### SUMMARY

Disclosed herein are compositions, methods, and systems for enhancing transport of cholesterol (including various modified cholesterol compounds) into modified mammalian mitochondria, wherein the modification renders the mitochondria capable of catabolizing cholesterol. In many embodiments, cells may be engineered for enhanced cholesterol flow across the mitochondrial membrane. The cells may also include one or more proteins that may aid in transport of cholesterol across the mitochondrial membrane.

Also disclosed are methods of making the disclosed compositions, and methods of using the disclosed compositions and methods for treating a subject having or at risk of developing high cholesterol, and may aid in reducing cholesterol in a subject. In various embodiments, the disclosed compositions may be administered to a subject in need of treatment for high cholesterol. In many embodiments, the nucleic acids expressing the disclosed proteins may be incorporated into a mammalian cell's genome or may be extra-genomic. The disclosed cells may be selected from a liver cell, neuronal cell, bone cell, muscle cell, endothelial cell, epithelial cell, immune cell, hematopoietic cell, and stem cell or precursor cell thereof.

Disclosed herein are compositions, methods, and systems for modifying mammalian mitochondria to enhance cholesterol transport and degradation. In many embodiments, mammalian mitochondria are engineered to comprise one or more recombinant and/or non-endogenous proteins that may help enhance cholesterol flow across the mitochondrial membrane. In many embodiments, the mitochondria may also include one or more proteins that may aid in catabolism of cholesterol. The disclosed modified mitochondria may be useful in reducing intracellular cholesterol concentrations, increasing cholesterol transport across the mitochondrial membrane, and/or reducing concentrations of cholesterol in the blood of a patient or subject at risk of developing, or suffering from high cholesterol or hypercholesterolemia.

In another aspect, a method of modifying a mammalian mitochondrion includes inserting at least one non-native engineered Steroidogenic Acute Regulatory (StAR) protein or portion thereof into the mammalian mitochondrion outer membrane, and where the mitochondrion includes one or more of a cholesterol degrading protein (CDP) includes cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).

In various aspects of the methods and compositions disclosed herein, the StAR protein may comprise residues 63 to 188 of SEQ ID NO:7, a duplication thereof, or at least a portion of the TOMM20 protein. The CDP may comprise P450 or P450-ferredoxin reductase-ferredoxin fusion protein (P450-FdxR-Fdx). The StAR protein and the CDP may comprise at least one mitochondrial signaling protein and/or be expressed from a cell-specific promoter sequence, and/or be expressed from a genome-integrated or from an extra genomic gene, and/or at least about 10% of the total amount of StAR protein and CDP in the cell may be located in a mitochondrial membrane. The disclosed methods and compositions may be useful in treating a subject suffering from or at risk of developing high cholesterol, such as by reducing cholesterol in the subject having high cholesterol. The cell may be selected from a liver cell, neuronal cell, bone cell, muscle cell, endothelial cell, epithelial cell, immune cell, hematopoietic cell, and stem cell or precursor cell thereof.

### BRIEF DESCRIPTION OF THE DRAWINGS

FIG. 1 depicts the mean catabolite concentration in culture media of cells expressing CDP in the presence and absence of StAR-StAR or Tom-StAR (n=3 independent experiments).

FIG. 2 is a representative western blot showing protein expression levels of tubulin, CDP, StAR-StAR, and TomStAR in cells of FIG. 1 (n=3 independent experiments).

FIG. 3 bar chart showing cholesterol degradation and consequent production of catabolite is greatly increased in vitro by the expression of StAR in mouse RAW264.7 cell lines expressing CDP.

FIG. 4 is a Western blot showing protein amounts in cell lines described in FIG. 3.

FIG. 5 are sequences of various proteins and nucleic acid coding sequences disclosed herein. Specifically, the Human protein sequence for p450-FdxR-Fdx (SEQ ID NO:1) is depicted with the Flag tag in bold; Cholesterol dehydrogenase, CholD (SEQ ID NO:2), is shown; n3-ketosteroid Δ1-dehydrogenase, Δ1-KstD (SEQ ID NO:3), is shown; Anoxic cholesterol metabolism B enzyme, acmB (SEQ ID NO:4) is shown; 3-ketosteroid 9α-hydroxylase, KshAB (SEQ ID NO:5) is shown; 3β-hydroxysteroid dehydrogenase 2, HSD2 (SEQ ID NO:6) is shown; Human StAR protein sequence (SEQ ID NO:7) is shown; Human TOMM20 protein sequence (SEQ ID NO:8) is shown; Human Tom/Star Fusion protein sequence (SEQ ID NO:9; Tom sequence is depicted in bold letters and the linker sequence is depicted in color and underlined) is shown; Human StAR/StAR fusion protein sequence (with linker sequence in color and underlined (SEQ ID NO:10); StAR mRNA complete sequence (SEQ ID NO:11; start codon shown capitalized) is depicted with the coding sequence corresponding to the underlined amino acids, 63-285, of SEQ ID NO 7 depicted in UPPERCASE lettering; and the TOMM20 mRNA complete sequence, CDS=123-560 (SEQ ID NO:12; start codon capitalized and stop shown in red) is also shown.

### DETAILED DESCRIPTION

Disclosed herein are compositions, methods, and systems for reducing cholesterol in a subject's cells and/or blood stream by enhancing transport of cholesterol into mitochondria where it is subjected to catalysis by cholesterol catabolizing enzymes.

Disclosed herein is the development of a unique approach to help manage hypercholesterolemia and other disorders related to the presence of excessive cholesterol. Humans lack enzymes to degrade cholesterol and many mammalian cells take up excess cholesterol without a way to property store or metabolize it. In some cases, macrophages progress to foam cells as they fill with cholesterol and cholesterol esters. This buildup of cholesterol may lead to formation of arterial plaques that can lead to atherosclerosis. Depending on their location, plaques may cause coronary artery disease, angina (chest pain), intermittent claudication (leg pain with exercise) or chronic kidney disease due to poor circulation. Moreover, many plaques can be unstable and prone to rupture even before they have any significant effect on blood flow. These structures are typically clinically silent until they rupture (complicated plaques) and predispose the underlying tissue to clots which may suddenly occlude the affected blood vessel.

Applicants show herein that human cells may be engineered to aid in reducing cholesterol. In many embodiments, the cells may be any mammalian cell. The disclosed cells may be engineered to express various proteins that aid in transport and degradation/catabolism of cholesterol. As used herein, an engineered cell may be a cell with one or more non-native and/or exogenous coding sequences or protein, for example an engineered mammalian cell may include one or more mammalian and/or bacteria-related sequences or proteins. Non-native, exogenous, or non-endogenous, as used herein may refer to a sequence or protein that is introduced to the cell, in most cases by recombinant methods. In most embodiments, bacteria-related may refer to a protein or coding sequence with greater than about 50% identity with a similar bacterial sequence. In many embodiments, the disclosed proteins may be fusion proteins and/or modified proteins, for example a protein lacking a sequence found in native proteins and/or duplicating sequences found in native proteins.

The disclosed proteins may be over-expressed relative to endogenous genes and/or proteins. In one example an endogenous gene or protein may be mammalian p450scc, StAR, or TOMM20 protein. In most embodiments, over-expression of the disclosed proteins may result in 2×, 3×, 4×, 5×, 10×, 20×, 100×, 200×, based on weight or moles, or more of the disclosed gene or protein in a given sample compared to the endogenous gene or protein. In most cases, overexpression is relative to non-induced expression of the endogenous protein. In some cases, over-expression may be relative to induced levels of endogenous protein, wherein expression may be similar or the same. In most embodiments, degradation in reference to degradation of cholesterol, refers to removal of various side chains and/or opening of at least one ring of cholesterol.

The disclosed coding sequences and proteins for cholesterol degradation may be derived from bacteria and/or mammals. In many embodiments, the disclosed proteins may be referred to as cholesterol degrading proteins (CDP) and may be selected from one or more of cholesterol dehydrogenase (CholD; SEQ ID NO:2), 3-ketosteroid Δ1-dehydrogenase (Δ1-KstD; SEQ ID NO:3), anoxic cholesterol metabolism B enzyme (acmB; SEQ ID NO:4), 3-ketosteroid 9α-hydroxylase (KshAB; SEQ ID NO:5), 3β-hydroxysteroid dehydrogenase 2 (HSD2; SEQ ID NO:6), a P450-ferredoxin reductase-ferredoxin fusion protein (P450-FdxR-Fdx; SEQ ID NO:1; uniprot numbers P05108, P22570, P10109), and steroidogenic acute regulatory protein (StAR; SEQ ID NO:7). In many embodiments, the disclosed coding sequences and proteins may share greater than about 80% identity to a sequence(s) disclosed herein.

The disclosed coding sequences and proteins may be delivered to the cell in-vivo or in-vitro, for example by various methods including electroporation, transfection, viral vector, nanoparticle, etc. In some embodiments, the disclosed coding sequences and proteins may be encoded by one or more nucleic acids within a cell, vector, or particle. In some embodiments, the particle may be lipid nanoparticle (or lipo nanoparticle; LNP). In some embodiments, the vector may be viral vector, such as adeno virus or lentivirus. In many embodiments, the coding sequences may include one or more promoter and/or enhancer sequences that may aid in expressing the coded proteins in a specific cell or tissue, or may aid in supporting high expression of the coded proteins, generally, or in a specific cell or tissue.

The disclosed proteins, which may be referred to collectively or singularly as cholesterol degrading proteins (CDPs) may be directed to the mitochondria of a mammal, for example the outer membrane, inner membrane, intermembrane space, or matrix.

The term “about” or“approximately” means an acceptable error for a particular value as determined by one of ordinary skill in the art, which depends in part on how the value is measured or determined. In certain embodiments, the term “about” or“approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

Administration of the disclosed compositions, constructs, cells, methods, and systems may be via various methods. In one embodiment, administration may be “intravenous” administration, referring to introducing the compositions, constructs, and/or cells into a vein of a patient, e.g. by infusion (slow therapeutic introduction into the vein). In other embodiments, administration may be “subcutaneous” wherein introduction is beneath the skin of the patient. “Infusion” or “infusing” refers to the introduction with a solution into the body through a vein for therapeutic purposes. Generally, this is achieved via an intravenous (IV) bag, such as a bag that can hold a solution. The disclosed compositions, constructs, cells, methods, and systems may be “co-administered” to a patient, for example by intravenously administering at least a second therapeutic agent during the same administration. In some embodiments, co-administration is concurrent, in others co-administration is sequential.

The term “prevention” as used herein means the avoidance of the occurrence or of the re-occurrence of a disease as specified herein, or at least one symptom associated therewith, by the administration of a composition, construct, method, or system according to the invention to a subject in need thereof.

A“patient” or“subject” includes various animals including a human, monkey, cat, dog, mouse, rat, rabbit or guinea pig. The animal can be a mammal such as a non-primate and a primate (e.g., monkey and human). In one embodiment, a patient is a human, such as a human infant, child, adolescent or adult.

The terms “treat”, “treating” and “treatment” refer to eliminating, reducing, suppressing, or ameliorating, either temporarily or permanently, either partially or completely, at least one symptom, manifestation, or progression of an event, disease, disorder, or condition disclosed herein. As is recognized in the pertinent field, methods, compositions, and systems employed as therapies may reduce the severity of a given disease state, but need not abolish every symptom associated therewith to be regarded as useful. Similarly, a prophylactically administered treatment need not be completely effective in preventing the onset of a condition to constitute a viable prophylactic composition, agent, method or system. Simply reducing the impact of a disease (for example, as disclosed herein, reducing blood cholesterol concentrations and/or reducing the number or severity of associated symptoms, or by increasing the effectiveness of another treatment, or by producing another beneficial effect), or reducing the likelihood that the disease will occur or worsen in a subject, is sufficient.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy or synergies with another therapeutic agent.

The phrase “therapeutically effective amount” means an amount of a composition of the present invention that (i) treats the particular disease, condition, or disorder, (ii) ameliorates or eliminates one or more symptoms of the particular disease, condition, or disorder, or (iii) prevents or delays the onset of one or more symptoms of the particular disease, condition, or disorder described herein. In the case of excess blood cholesterol, the therapeutically effective amount of the disclosed compositions may reduce the concentration of cholesterol in the patient's blood; reduce intercellular cholesterol; inhibit (i.e., slow to some extent and preferably stop) cholesterol plaque growth and/or development; inhibit (i.e., slow to some extent and preferably stop) lipid buildup in the liver; reduce lipid content in the liver; and/or relieve to some extent one or more of the symptom associated with the high concentrations of blood cholesterol.

The term “amelioration” as used herein refers to any improvement of a disease state (for example high blood cholesterol) of a patient, by the administration of one or more treatments and/or compositions, according to the present disclosure, to such patient or subject in need thereof. Such an improvement may be seen as a slowing down or stopping the progression of the disease of the patient, and/or decreasing the severity of at least one disease symptom, an increase in frequency or duration of disease symptom-free periods or a prevention of impairment or disability due to the disease.

### Target Cells

Various tissues and cell types made to express CDP may be targeted with the disclosed compositions. In various embodiments, the cells may be endothelial and epithelial cells in all organs and tissues, including, without limitation, smooth, skeletal and cardiac muscle cells, neuronal cells, glandular cells, bone cells, immune cells, hematopoietic cells, and stem cells or precursor cells thereof. In some embodiments, the cell maybe a pluripotent stem cells, for example an induced pluripotent stem cell. In many embodiments, the tissues may be cancerous or pre-cancerous, and may one or more cells of skin and intestinal carcinomas (squamous epithelial cells), adenocarcinoma (epithelial cells of glandular origin, pancreatic) gastrointestinal carcinoid (neuroendocrine cells), pancreatic cancer, small cell carcinoma (neuroendocrine cells), leiomyosarcoma (smooth muscle cells) and lymphomas (lymphocytes found in organs/walls of the gastrointestinal tract).

Various cells and tissues may be targeted with the disclosed compositions and methods. In many embodiments, the disclosed compositions may be targeted to specific cells by various methods known to those of skill in the art. In some embodiments, LNPs may include one or more molecules that help target specific cells, for example a protein with affinity for a receptor or membrane protein on the target cell.

### Cholesterol Degrading Proteins

Disclosed herein are various cholesterol, and cholesterol-related genes and proteins. In some embodiments, the disclosed genes and proteins may aid in transport and/or metabolism of cholesterol. In most embodiments the disclosed genes and proteins may be expressed in and/or targeted to the mitochondria. In many embodiments the disclosed genes and proteins may allow cells to degrade and/or catabolize cholesterol to ring opening, whereupon endogenous proteins and enzymes may further metabolize the molecule.

### Mitochondria

Mitochondria are found in most eukaryotic cells and organisms, and consist of a double membrane. Mitochondria supply the cell's energy, and play a role in signaling, cell-cycle, cell growth and differentiation, apoptosis, and cell death. Cells with high demand for energy, such as heart, muscle, brain, and liver cells, may have several thousand mitochondria.

The double-membrane structure of the mitochondria allows for specialized compartments: the outer membrane, the intermembrane space, the inner membrane, and the cristae and matrix. These compartments allow for specialized functions—such as ATP synthesis. The matrix is home to the mitochondrion's independent genome.

### Steroidogenic Acute Regulatory Protein (StAR)

Steroids and steroidal hormones are synthesized from cholesterol. The first step in steroidogenesis is the cleavage of cholesterol's side chain by P450scc, which is located on the matrix side of the inner mitochondrial membrane (IMM). Typically, cells that produce steroid hormones store and possess only small amounts of hormone at any one time. Thus, increases in steroid secretion is accomplished by first increasing synthesis of the steroid. However, steroids have broad and powerful action within the cell and organism—thus steroidogenic cells tightly regulate production of steroids and precursors to avoid overproduction and pathologies associated with excess amounts of steroid. Synthesis can be rapidly inducted and rapidly terminated to avoid overproduction. One way cells exert this control (which can increase synthesis 10-100-fold within minutes) is to regulate the flow of cholesterol into the mitochondrial matrix. Cholesterol is transported across the mitochondrial membrane by the StAR protein.

The StAR protein is synthesized as a 37 kDa protein comprising a mitochondrial targeting leader peptide sequence. Upon insertion into the mitochondrial membrane, the leader peptide is cleaved to yield the 30 kDa protein that is located intra-mitochondrially. Mutations in the StAR gene can result in congenital lipoid adrenal hyperplasia. Subjects suffering from adrenal hyperplasia synthesize very small amounts of steroid.

StAR may interact with two additional proteins to support importation of cholesterol into mitochondria. TSPO, Translocator Protein, is an 18 kDa protein that aids mitochondrial import of steroidogenic cholesterol. Studies have shown that TSPO expression is upregulated in response to exposure of macrophages to modified LDLs. In addition, the ubiquitously expressed PBR (peripheral-type benzodiazepine receptor) is an outer mitochondrial membrane protein involved in regulating cholesterol transport. Inhibition of PBR results in loss of a cells steroidogenic capacity.

The ability to produce large amounts of steroids from cholesterol can be imparted to non-steroidogenic cells (i.e. cells that do not typically produce steroids or produce very low amounts of steroids) by introducing StAR. Specifically, non-steroidogenic cells transfected with the P450scc enzyme system (i.e. H₂N-P450scc-adrenodoxin reductase-adrenodoxin-COOH) are able to metabolize cholesterol at very low levels. The presence of StAR (i.e. co-expression of StAR and P450scc enzyme system) increases conversion of cholesterol several fold in these cells. Studies have shown that the cells' production of steroids can be further enhanced by anchoring StAR in the outer mitochondrial membrane (OMM), either by fusion to an OMM protein (e.g. the mitochondrial translocase TOM gene translocase, outer membrane) or duplication of StAR domain, which may help to slow transit of StAR through the OMM. Duplication of the StAR domain may be referred to as StAR-StAR protein. StAR-StAR is a fusion protein that combines 1-188 residues of StAR to 63-285 residues of StAR, thus dimerizing the protease-resistant domain of StAR (residues 63-188).

StAR proteins for use with the present compositions and methods may include various forms of the StAR protein sequence. In some embodiments, the disclosed StAR proteins may include one or more mutations, truncations, deletions, duplications, fusions, etc. or the disclosed proteins may be wild-type or native StAR proteins. In some embodiments, the disclosed StAR proteins may be modified to reduce transit time through the outer membrane, which may help to enhance mitochondrial import of cholesterol.

As used herein, the term cholesterol refers to cholesterin or cholesteryl alcohol, a sterol of formula C₂₇H₄₆O, with IUPAC names cholest-5-en-3β-ol, and (3S,8S,9S,10R,13R,14S,17R)-10,13-dimethyl-17-[(2R)-8-methylheptan-2-yl]-2,3,4,7,8,9,11,12,14,15,16,17-dodecahydro-1H-cyclopenta[a]phenanthren-3-ol. As used herein, the term cholesterol may also refer to derivatives of cholesterol, including oxidized cholesterol, C₂₇H₄₆O₂, Oxycholesterol, or 5,6-epoxycholesterol, 7-ketocholesterol (7KC), cholestane-3β,5α,6β-triol and 7-α/β hydroxycholesterol, etc.

The disclosed genes and proteins that may degrade cholesterol may be selected from cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

One or more of the disclosed genes, proteins, and enzymes may be packaged into one or more vector, construct, or cassette. In various embodiments, a cassette that includes one or more cholesterol degrading enzymes may be referred to as a cholesterol catabolizing cassette (CCC). In some embodiments the cassette may be a construct and may include a nucleic acid sequence that codes for at least one protein that is about 80% or more identical to a protein disclosed herein or a protein coded for by any of the disclosed genes. In many embodiments, the percent identity with the disclosed protein or nucleotide sequences may be greater than about 79%, 80%, 81%, 82%, 83%, 84%, 85%, 86%, 87%, 88%, 89%, 90%, 91%, 92%, 93%, 94%, 95%, 96%, 97%, 98%, or 99%, and less than about 100%, 99%, 98%, 97%, 96%, 95%, 94%, 93%, 92%, 91%, 90%, 89%, 88%, 87%, 86%, 85%, 84%, 83%, 82%, and 81%. Sequence identity may be calculated for a protein sequence of greater than about 40 aa (amino acid residues), 50 aa, 60 aa, 70 aa, 80 aa, 90 aa, 100 aa, 110 aa, 120 aa, 130 aa, 140 aa, 150 aa, 160 aa, 170 aa, 180 aa, 190 aa, 200 aa or more and less than about 300 aa, 200 aa, 190 aa, 180 aa, 170 aa, 160 aa, 150 aa, 140 aa, 130 aa, 120 aa, 110 aa, 100 aa, 90 aa, 80 aa, 70 aa, or 60 aa. In many embodiments, sequence identity is calculated over about 80-150 amino acid residues. In most cases an inserted or deleted amino acid, compared to the reference sequence (i.e. SEQ ID NOs: 1-10), counts as a single non-identical amino acid. In some embodiments, for example wherein delivery of the one or more compositions is via an LNP, for example where the composition is one or more nanoplasmid or mRNA, the composition may include one or more cholesterol degrading proteins, for example an enzyme(s) and a cholesterol transporting protein, for example StAR protein or StAR variant. One of skill in the art is capable of creating an mRNA from the protein and nucleic acid sequences disclosed herein. In embodiments where the one or more compositions are comprised in a therapeutic cell that may be administered to a subject, the therapeutic cell may be administered by one or more vectors, constructs, and/or cassettes comprising nucleic acids that code for a cholesterol degrading protein, for example an enzyme and a cholesterol transport protein, for example StAR or a StAR variants. In these cases, the nucleic acids may be transduced into the cell, for example by one or more viral vectors (in one example lentivirus) to aid in integrating or inserting the coding sequence into the genomic sequence or mitochondrial sequence of the cell. In some embodiments, the nucleic acids may not be integrated, and the disclosed proteins are expressed from extra-genomic sequences in the nucleus or mitochondria.

### Cholesterol-Related Diseases and Disorders

Excess native LDL and increased LDL:HDL ratio have been shown to play critical roles in cardiovascular disease, atherosclerosis, stroke and coronary heart disease and heart attacks. In some embodiments, a subject or patient at risk for, or suffering from, high cholesterol may have total cholesterol concentration of greater than about 200 mg of cholesterol (i.e. low-density lipoprotein+high-density lipoprotein) per decilitre of blood and/or more than about 5.2 mmol/L total cholesterol. In some embodiments, a subject or patient at risk for, or suffering from, high cholesterol may have an LDL concentration of greater than about 100 mg/dl and/or more than about 2.6 mM LDL. In some embodiments, a subject or patient at risk for, or suffering from, high cholesterol may have an HDL concentration of less than about 50 mg/dl and/or less than about 1.3 mM HDL. In some embodiments, a subject or patient at risk for, or suffering from, high cholesterol may have a triglyceride concentration of greater than about 150 mg/dl and/or greater than about 1.7 mM triglycerides. In some embodiments, a subject or patient at risk for, or suffering from, high cholesterol may have an LDL:HDL ratio of about 4.0 (i.e. 4 to 1) or more than 4.0.

Acetylated LDL is an in vitro chemically modified form of LDL and does not exist in vivo. Both acetylated LDL and oxidized LDL, are taken up by macrophages, transforming those cells into foam cells. In most cases, all components of LDL are susceptible to oxidation, producin an oxidized form of LDL (oxLDL). The uptake of oxLDL by arterial macrophages is pivotal in the formation of plaques. Unlike unmodified LDL, oxLDL is taken up by arterial wall macrophages in an unregulated manner via LDL scavenger receptors. Oxysterols are 10-100× more reactive than native cholesterol, with the most toxic of these being 7-ketocholesterol (7KC), which is also the most abundant in oxLDL.

Studies find that higher levels of circulating 7KC are associated with greater future risk of cardiovascular events and increased total mortality. 7KC is a pro-inflammatory, pro-oxidant, pro-apoptotic, and fibrogenic molecule that alters endothelial cell function by disrupting cell membranes and critical ion transport pathways for vasodilatory response. In some embodiments, a subject or patient at risk for, or suffering from, high cholesterol may have a 7KC concentration higher than about 109.8 nmol/L nmol/L.

A subject or patient suffering from, or at risk of developing high cholesterol may be referred to as hypercholesterolemic. In some embodiments, hypercholesterolemia may be assessed by measuring the percentage of plasma oxysterols, for example the percentage of 7-KC in plasma oxysterols. In hypercholesterolemic patients, 7 KC may account for about 57% of the plasma oxysterols. 7KC is followed by 7-α/β hydroxycholesterol (at 21% of plasma oxysterols), which is a direct product of 7KC metabolism. In arterial plaques, 55% of oxysterols are reported to be 7KC, with the second and third most abundant being cholestane-3β,5α,6β-triol and 7-α/β hydroxycholesterol at 13% and 12%, respectively.

As noted above, NASH (nonalcoholic steatohepatitis) is another disease associated with excess cholesterol. Altered cholesterol homeostasis and transport contribute to the accumulation of free cholesterol in the liver, which in turn contributes to NAFLD (Non-alcoholic fatty liver disease) via damage to hepatocytes and the activation of non-parenchymal cells. Particularly, the overload of free cholesterol in and around the mitochondria induces mitochondrial dysfunction and promotes inflammation, fibrosis and hepatocyte death.

Other cholesterol-associated diseases include pulmonary alveolar proteinosis (PAP), eye disease, neurodegenerative diseases, Niemann Pick Type C (NPC), and Lysosomal Acid Lipase (LAL) deficiency. Because cholesterol content plays a role in regulating surfactant fluidity and function in lunged animals, and that fluidity can change rapidly, especially under extremes of temperature, reduced cholesterol clearance is a primary defect driving PAP pathogenesis. In the case of eye disease, oxysterols and, in particular 7KC, cause degeneration of retinal cells. Thus, increased oxysterol levels may play a role in various eye diseases including macular degeneration (AMD), choroidal neovascularization (CNV), glaucoma, and cataracts.

Increased oxysterol levels may also result in alterations in brain cholesterol metabolism. Cholesterol metabolism may be an integral part of several brain disorders including Alzheimer's disease, Amyotrophic Lateral Sclerosis (ALS), Parkinson's disease, and dementia progression. Various oxysterols derived from the auto-oxidation of cholesterol, including 7KC have been identified in post-mortem brains of patients with Alzheimer's disease. Chronic epilepsy may also share many of these pathologies. Specifically, a link has been suggested between epilepsy and atherosclerosis. Thus, treatment of atherosclerosis, such as the presently disclosed compositions, cells, and methods may lessen the effects of epilepsy. Further, 7KC is highly cytotoxic to neuronal cells and has been suspected to be involved in the progression of various neurological diseases. Surprisingly, oxysterols, unlike cholesterol, can cross the blood brain barrier (BBB) and accumulate in brain tissue, ultimately causing neurodegeneration.

Various other diseases may be linked with increased cholesterol levels. For example, patients with Niemann Pick Type C (NPC) are inable to clear cholesterol, causing the accumulation of cholesterol and oxysterols in mostly the liver, spleen, and brain. A positive correlation between the 7KC profile and the severity of the disease has been reported. In addition, patients with Lysosomal Acid Lipase (LAL) deficiency accumulate cholesterol esters and triglycerides in lysosomes, and can present with hypercholesterolemia, hyperlipidemia, and/or atherosclerosis. These patients also have very high levels of oxysterols, including 7KC, in their plasma. Increased formation of oxysterols further increases oxidative stress worsens the condition.

The disclosed compositions and methods are useful in treating diseases or conditions associated with excess cholesterol and/or fat deposits in cells, tissues, and organs. In some embodiments, the disease or condition may be associated with excess cholesterol and/or the presence of one or more oxidized cholesterol species, such as 7-ketocholesterol. In some embodiments, the disease or condition may be one or more of fatty liver disease, atherosclerosis, heart failure, stroke, ischemia, coronary heart disease, eye disease, neurodegenerative and neurological disease, diseases of the eye, such as macular degeneration, pulmonary dysfunction, etc.

The disclosed compositions, cells, methods, and therapies may aid in treating, reducing, or reversing various diseases, disorders, or conditions related to excess cholesterol. In one embodiment, the disease, disorder, or condition may be one or more of early type II lesions (i.e. macrophage foam cell formation), type III lesions or pre-atheromas (i.e. having small pools of extracellular lipids), type IV lesions or atheromas (i.e. having a core of extracellular lipids), type V lesions or fibroatheromas (i.e. atheromas with fibrous thickening).

The disclosed compositions, constructs, cells, methods, therapies, and systems may aid in reducing the concentration of cholesterol in the blood of a patient. In some embodiments, a decrease in the concentration of cholesterol in the blood (e.g. weight per volume, such as mg/dl, or molarity, such as mmol/L) may be greater than about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% and less than about 100%, 95%, 90%, 80%, 70%, 60%, 50%, 40%, 30%, 20%, 10%, 9%, 8%, 7%, 6%, 5%, 4%, 3%, or 2%. In many embodiments, the decrease in cholesterol levels upon treatment may be reflected in an increase in the concentration or amount of one or more cholesterol catabolites, for example an increase of greater than 10%, 20%, 30%, 40%, 50%, 60%, 70%, 80%, 90%, 100%, 1.5×, 2.0×, 2.5×, 3.0×, 3.5×, 4.0×, 4.5×, 5.0×, 10×, 20×, 30×, 40×, 50×, 60×, 70×, 80×, 90×, 100×, 200×, 300×, 400×, 500×, 600×, 700×, 800×, or more, and less than about 2000×, 1500×, 1000×, 500×, 400×, 300×, 200×, 100×, 90×, 80×, 70×, 60×, 50×, 40×, 30×, 20×, 10×, 5×, 4×, 3×, 2×, 100%, 90%, 80%, 70%, 60%, 50%, 40%, 30%, 20%, or 10% over the concentration of catabolite prior to treatment.

### EXAMPLES

### Example 1—Co-Expression of Cholesterol Degrading Enzymes and StAR Protein

In vitro experiments were performed to test the cholesterol degrading activity of cholesterol degrading proteins in combination with StAR. In these experiments, Human 293T kidney epithelial cells were seeded at 3×10⁵ 293T cells/well in a 12-well plate in DMEM and 10% FBS. At 75% confluency, cells were transfected Lipofectamine 3000. Next, equimolar amounts of a CDP or empty plasmid and either a StARStAR, TomSTaR, or empty plasmid were mixed with P3000 in 50 μL of Opti-MEM to have the following conditions:
1. Empty plasmid+Empty plasmid
2. CDP plasmid+Empty plasmid
3. StARStAR plasmid+Empty plasmid
4. TomStAR plasmid+Empty plasmid
5. CDP plasmid+StARStAR plasmid
6. CDP plasmid+TomStAR plasmid

3× μL of Lipofectamine Reagent was mixed with 50 μL of Opti-MEM. Next, the DNA/P3000 tube was combined with the Lipofectamine tube and incubated at room temperature for 15 minutes. The transfection mixture was added in a dropwise fashion to the cells and incubated for 48 hours.

At the end of the incubation period, the cell media was assayed for catabolite concentration. Total cellular protein was extracted using RIPA buffer and total protein concentrations were quantified using a standard BCA kit.

### Results:

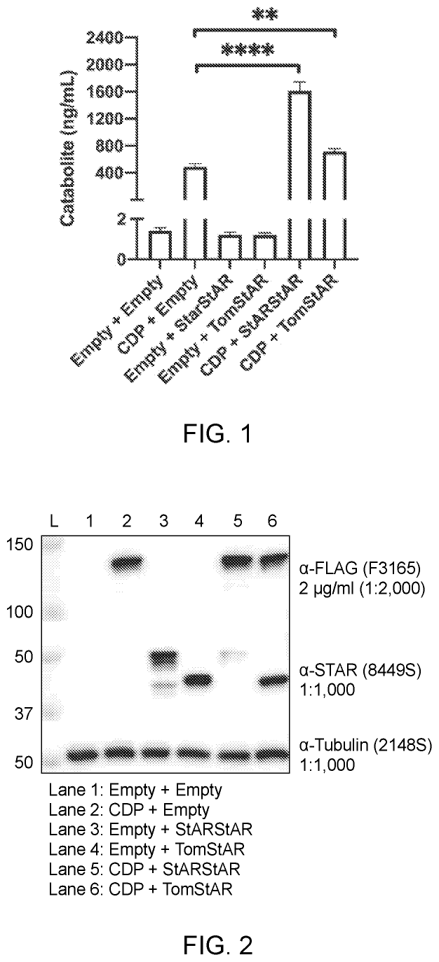

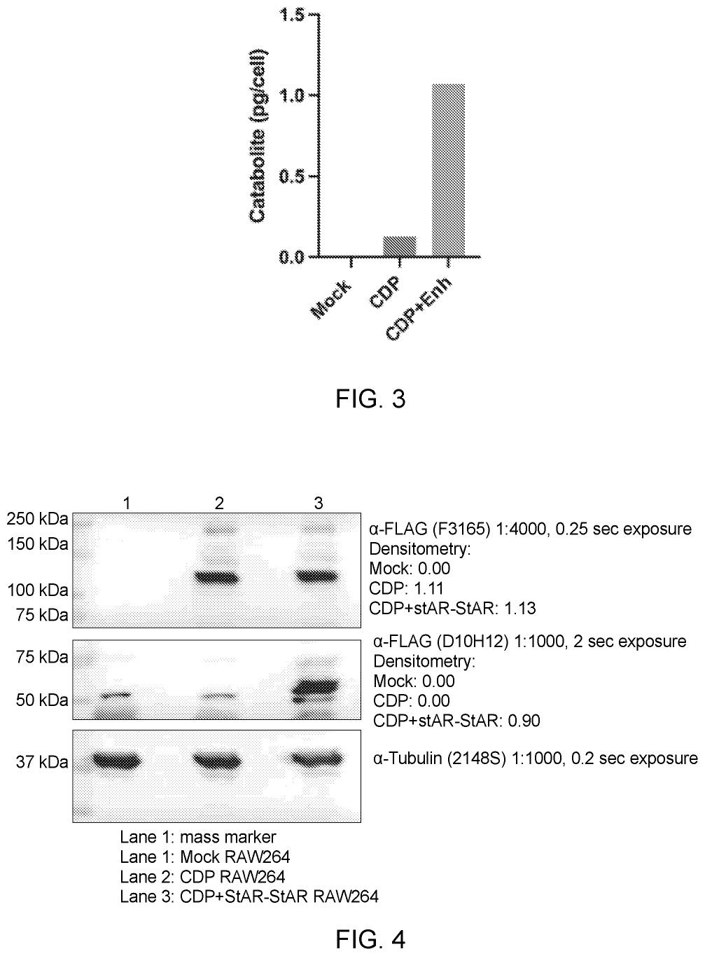

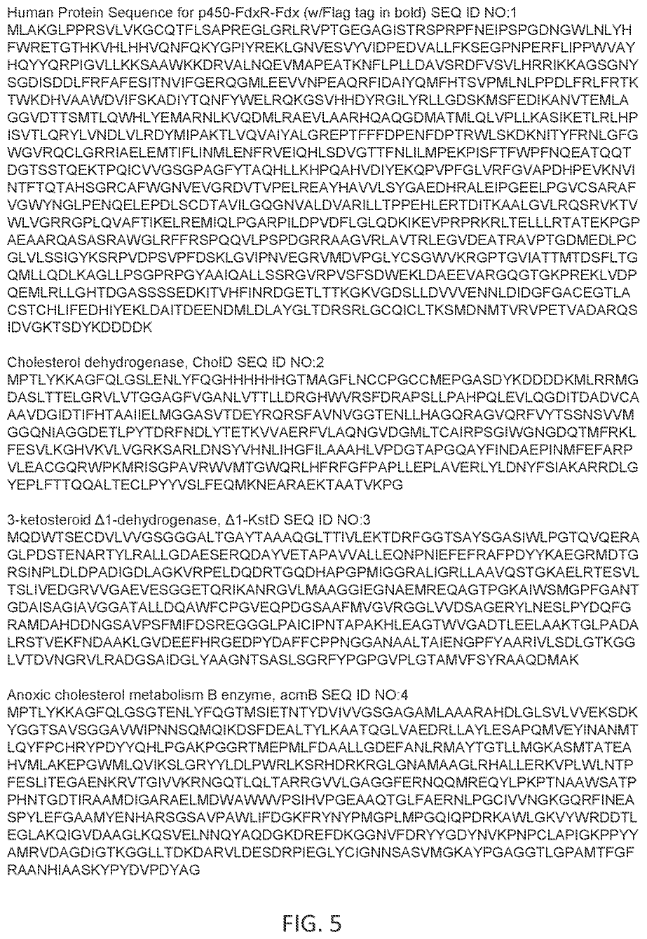

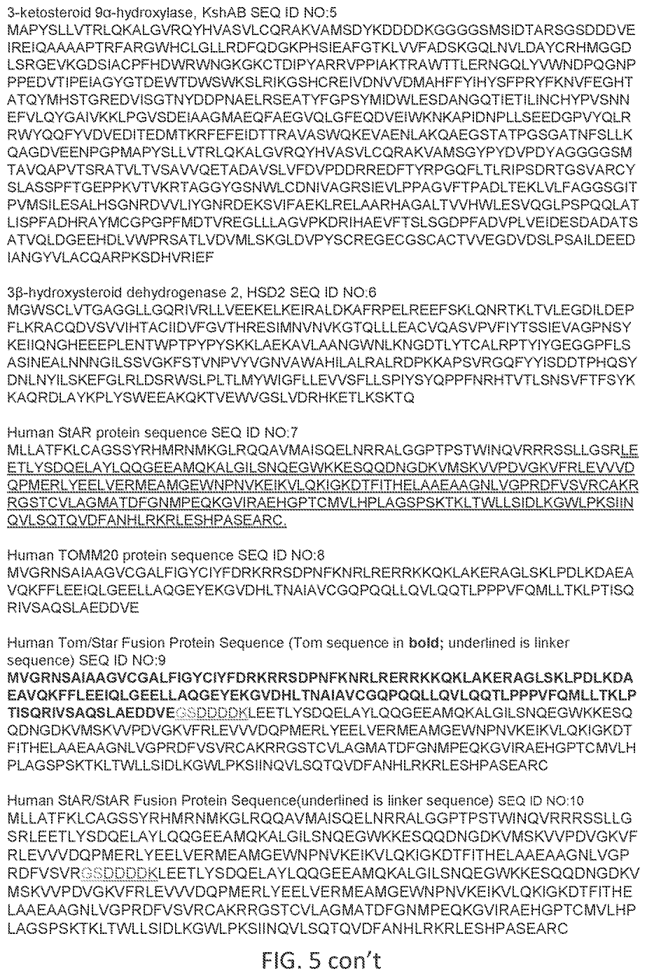

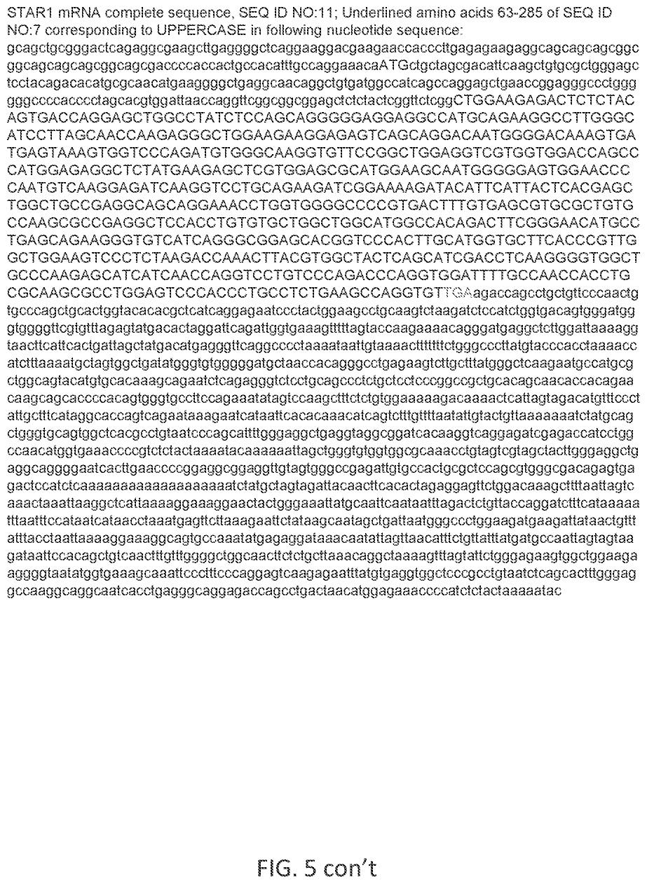

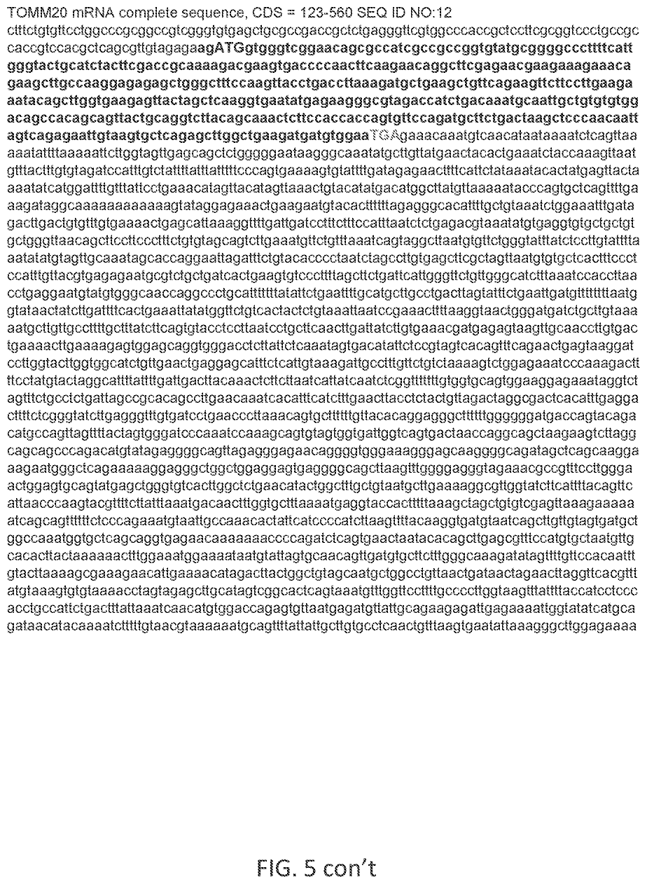

As shown in FIG. 1, below, the expression of CDP in 293T kidney epithelial cells resulted in a ˜300-fold increase catabolite concentration in culture media from baseline levels (media of cells expressing only empty plasmid i.e. no CDP). Co-expression of this fusion protein with CDP increased catabolite production ˜4-fold compared to CDP alone. Tom20 has 50N-terminal residues embedded in the outer mitochondrial membrane (OMM) and 95 residues in the cytoplasm. Fusion of StAR residues 63-285 to the carboxyl terminus of Tom20 (i.e. Tom-StAR) affixes StAR to the cytoplasmic side of the OMM and, when co-expressed with CDP, increased catabolite production by ˜30% compared to cells expressing CDP alone. Expression of either StAR-StAR or TomStAR alone did not affect catabolite concentrations. Transfection of cells with StAR-StAR cDNA resulted in robust expression of the StAR-StAR protein detected as a ˜50 kD band by SDS-PAGE and western blot, see FIG. 2, below. Interestingly, co-expression of CDP (detected as a FLAG-tagged protein with anti-FLAG antibody) with StAR-StAR resulted in a significant downregulation of steady-state StAR-StAR protein levels. Without wishing to be bound to a theory, these data suggest that continued StAR-StAR-mediated transport of the CDP-substrate, cholesterol, into the mitochondrial matrix result in the destabilization and degradation/consumption of StAR-StAR protein. Downregulation of Tom-StAR was not detected in cells co-expressing CDP and was perhaps due, without wishing to be bound to a theory, to the lower efficiency of Tom-StAR in transporting cholesterol to the mitochondrial matrix. This reduced efficiency was evidenced by significantly lower levels of CDP-derived catabolite in CDP and Tom-StAR co-expressing cells versus those co-expressing CDP and StAR-StAR (see FIG. 1 above). The level of StAR-StAR protein was higher in cells expressing only StAR-StAR compared to those expressing StAR-StAR and CDP, suggesting that the level of StAR-StAR activity and its consequent degradation might be limited by increased levels of cholesterol accumulation in the mitochondrial matrix in the absence of CDP. We speculate that continued conversion of cholesterol by CDP to its catabolite increases the cholesterol concentration gradient along the inner mitochondrial membrane, thus further stimulating StAR-StAR-mediated cholesterol transport into the mitochondrion.

FIG. 1 shows results from this experiment in bar graph format. Specifically, FIG. 1 shows the mean catabolite concentration in media of cells expressing CDP in the presence and absence of StAR-StAR or Tom-StAR (n=3 independent experiments) as indicated below the graph.

FIG. 2 is a representative western blot showing CDP protein levels in cells in the presence and absence of StAR-StAR or Tom-StAR with tubulin as control (n=3 independent experiments). Lanes are as follows: 1, Empty plasmid+Empty plasmid; 2, CDP plasmid+Empty plasmid; 3, StARStAR plasmid+Empty plasmid; 4, TomStAR plasmid+Empty plasmid; 5, CDP plasmid+StARStAR plasmid; 6, CDP plasmid+TomStAR plasmid.

### Example 2—Co-Expression of Cholesterol Degrading Enzymes and StAR Protein in Mouse Cells

In vitro experiments were performed as described in Example 2. In these experiments, Mouse RAW264.7 cells were seeded at 3×10⁵ 293T cells/well in a 12-well plate in DMEM and 10% FBS. At 75% confluency, cells were transfected Lipofectamine 3000. Next, equimolar amounts of a CDP or empty plasmid and either a StARStAR or empty plasmid were mixed with P3000 in 50 μL of Opti-MEM to have the following conditions: Empty plasmid+Empty plasmid; CDP plasmid+Empty plasmid; or CDP plasmid+StARStAR plasmid.

Cholesterol catabolism greatly increased in cells co-expressing CDP and StAR. Specifically catabolite production, in pg/cell were as follows: Empty+Empty, 2.83E-03; CDP+Empty, 1.26E-01; and CDP+StARStAR, 1.07E+00. These results correspond to a 44.52-fold increase in the presence of CDP, and an additional 8.49-fold increase in the presence of StARStAR protein.
Claim 1. A nucleic acid composition comprising:
a coding sequence for Steroidogenic Acute Regulatory (StAR) protein; and
a coding sequence for a cholesterol degrading protein (CDP) comprising one or more of cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 2. The nucleic acid of claim 1, wherein the StAR protein comprises residues 63 to 188 of SEQ ID NO:7.
Claim 3. The nucleic acid of claim 1 or claim 2, wherein the StAR protein comprises a duplication of residues 63-188 of SEQ ID NO:7.
Claim 4. The nucleic acid of any of claim 1 to claim 3, wherein the StAR protein comprises at least a portion of the TOMM20 protein.
Claim 5. The nucleic acid of any of claim 1 to claim 4, wherein the CDP comprises P450.
Claim 6. The nucleic acid of any of claim 1 to claim 5, wherein the CDP comprises P450-ferredoxin reductase-ferredoxin fusion protein (P450-FdxR-Fdx).
Claim 7. The nucleic acid of any of claim 1 to claim 6, wherein the StAR protein and the CDP comprise at least one mitochondrial signaling protein.
Claim 8. The nucleic acid of any of claim 1 to claim 7, wherein the nucleic acid further includes a cell-specific promoter sequence.
Claim 9. The nucleic acid of any of claim 1 to claim 8, wherein the nucleic acid is comprised in a lipo nanoparticle.
Claim 10. The nucleic acid of any of claim 1 to claim 8, wherein the nucleic acid is comprised in a viral vector.
Claim 11. The nucleic acid of any of claim 1 to claim 10, for use in treating a subject having high cholesterol.
Claim 12. The nucleic acid of any of claim 1 to claim 11, for use in transforming a mammalian cell.
Claim 13. The nucleic acid of claim 12, wherein the mammalian cell is transformed in-vitro.
Claim 14. The nucleic acid of claim 12, wherein the mammalian cell is transformed in-vivo.
Claim 15. The nucleic acid of any of claim 1 to claim 14, wherein the nucleic acid is comprised in a lipo nanoparticle.
Claim 16. The nucleic acid of any of claim 1 to claim 15, wherein the nucleic acid is comprised in a cell of subject suffering from or at risk of developing high cholesterol.
Claim 17. The nucleic acid of any of claim 1 to claim 16, for use in reducing cholesterol in a subject having high cholesterol.
Claim 18. A mammalian cell comprising:
a coding sequence for an engineered Steroidogenic Acute Regulatory (StAR) protein or portion thereof; and
a coding sequence for a cholesterol degrading protein (CDP) comprising one or more of cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 19. The mammalian cell of claim 18, wherein the StAR protein comprises residues 63 to 188 of SEQ ID NO:7.
Claim 20. The nucleic acid of claim 18 or claim 19, wherein the StAR protein comprises a duplication of residues 63-188 of SEQ ID NO:7.
Claim 21. The mammalian cell of any of claim 18 to claim 20, wherein the StAR protein comprises at least a portion of a TOMM20 protein sequence.
Claim 22. The mammalian cell of any of claim 18 to claim 21, wherein the CDP comprises P450.
Claim 23. The mammalian cell of any of claim 18 to claim 22, wherein the CDP comprises P450-ferredoxin reductase-ferredoxin fusion protein (P450-FdxR-Fdx).
Claim 24. The mammalian cell of any of claim 18 to claim 23, wherein the StAR protein and the CDP comprise at least one mitochondrial signaling protein.
Claim 25. The mammalian cell of any of claim 18 to claim 24, wherein the coding sequence of the StAR or CDP further includes a cell-specific promoter sequence.
Claim 26. The mammalian cell of any of claim 18 to claim 25, wherein the coding sequence of the StAR or CDP is integrated into the mammalian cell's genome.
Claim 27. The mammalian cell of any of claim 18 to claim 25, wherein the coding sequence of the StAR or CDP is not integrated into the genome of the mammalian cell.
Claim 28. The mammalian cell of any of claim 18 to claim 27, for use in treating a subject suffering from or at risk of developing high cholesterol.
Claim 29. The mammalian cell of any of claim 18 to claim 28, for use in reducing cholesterol in a subject.
Claim 30. The mammalian cell of any of claim 18 to claim 29, wherein the mammalian cell is selected from a liver cell, neuronal cell, bone cell, muscle cell, endothelial cell, epithelial cell, immune cell, hematopoietic cell, and stem cell or precursor cell thereof.
Claim 31. A mammalian cell comprising:
an engineered Steroidogenic Acute Regulatory (StAR) protein or portion thereof; and
a cholesterol degrading protein (CDP) comprising one or more of cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 32. The mammalian cell of claim 31, wherein the StAR protein comprises residues 63 to 188 of SEQ ID NO:7.
Claim 33. The mammalian cell of claim 31 or claim 32, wherein the StAR protein comprises a duplication of residues 63-188 of SEQ ID NO:7.
Claim 34. The mammalian cell of any of claim 31 to claim 33, wherein the StAR protein comprises at least a portion of the TOMM20 protein.
Claim 35. The mammalian cell of any of claim 31 to claim 34, wherein the CDP comprises P450.
Claim 36. The mammalian cell of any of claim 31 to claim 35, wherein the CDP comprises P450-ferredoxin reductase-ferredoxin fusion protein (P450-FdxR-Fdx).
Claim 37. The mammalian cell of any of claim 31 to claim 36, wherein the StAR protein and the CDP comprise at least one mitochondrial signaling protein.
Claim 38. The mammalian cell of any of claim 31 to claim 37, wherein at least 10% of the total amount of StAR protein and CDP in the cell are located in a mitochondrial membrane.
Claim 39. The mammalian cell of any of claim 31 to claim 38, wherein the StAR protein or the CDP is coded for by a gene integrated into the genome of the mammalian cell.
Claim 40. The mammalian cell of any of claim 31 to claim 39, wherein the StAR protein or the CDP is coded for by an extra genomic gene.
Claim 41. The mammalian cell of any of claim 31 to claim 39, for use in treating a subject suffering from or at risk of developing high cholesterol.
Claim 42. The mammalian cell of any of claim 31 to claim 40, for use in reducing cholesterol in a subject.
Claim 43. The mammalian cell of any of claim 18 to claim 29, wherein the cell is selected from a liver cell, neuronal cell, bone cell, muscle cell, endothelial cell, epithelial cell, immune cell, hematopoietic cell, and stem cell or precursor cell thereof.
Claim 44. A method of enhancing cholesterol degradation in a mammalian cell comprising:
expressing non-native engineered Steroidogenic Acute Regulatory (StAR) protein or portion thereof; and
expressing one or more of a cholesterol degrading protein (CDP) comprising cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 45. The method of claim 44, wherein the StAR protein comprises residues 63 to 188 of SEQ ID NO:7.
Claim 46. The method of claim 44 or claim 45, wherein the StAR protein comprises a duplication of residues 63-188 of SEQ ID NO:7.
Claim 47. The method of any of claim 44 to claim 46, wherein the StAR protein comprises at least a portion of the TOMM20 protein.
Claim 48. The method of any of claim 44 to claim 47, wherein the CDP comprises P450.
Claim 49. The method of any of claim 44 to claim 48, wherein the CDP comprises P450-ferredoxin reductase-ferredoxin fusion protein (P450-FdxR-Fdx).
Claim 50. The method of any of claim 44 to claim 49, wherein the StAR protein and the CDP comprise at least one mitochondrial signaling protein.
Claim 51. The method of any of claim 44 to claim 50, wherein the StAR protein and CDP are expressed from a cell-specific promoter sequence.
Claim 52. The method of any of claim 44 to claim 51, for use in treating a subject suffering from or at risk of developing high cholesterol.
Claim 53. The method of any of claim 44 to claim 52, for use in reducing cholesterol in a subject having high cholesterol.
Claim 54. The method of any of claim 44 to claim 53, wherein at least about 10% of the total amount of StAR protein and CDP in the cell is located in a mitochondrial membrane.
Claim 55. The method of any of claim 44 to claim 54, wherein the StAR protein or the CDP is expressed from a gene integrated into the mammalian cell's genome.
Claim 56. The method of any of claim 44 to claim 54, wherein the StAR protein or the CDP is from an extra genomic gene.
Claim 57. The method of any of claim 44 to claim 56, for use in treating a subject suffering from or at risk of developing high cholesterol.
Claim 58. The method of any of claim 44 to claim 57, for use in reducing cholesterol in a subject.
Claim 59. The method of any of claim 44 to claim 58, wherein the cell is selected from a liver cell, neuronal cell, bone cell, muscle cell, endothelial cell, epithelial cell, immune cell, hematopoietic cell, and stem cell or precursor cell thereof.
Claim 60. The method of any of claim 44 to claim 59, wherein the cholesterol is LDL.
Claim 61. The method of any of claim 44 to claim 59, wherein the cholesterol is 7KC.
Claim 62. A method of treating a patient at risk of developing or suffering from high cholesterol comprising:
administering to the patient at least one modified cell, the modified cell comprising
a non-native engineered Steroidogenic Acute Regulatory (StAR) protein or portion thereof; and
one or more of a cholesterol degrading protein (CDP) comprising cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 63. The method of claim 62, wherein the StAR protein comprises residues 63 to 188 of SEQ ID NO:7.
Claim 64. The method of claim 62 or claim 63, wherein the StAR protein comprises a duplication of residues 63-188 of SEQ ID NO:7.
Claim 65. The method of claim 62 to claim 64, wherein the StAR protein comprises at least a portion of the TOMM20 protein.
Claim 66. The method of claim 62 to claim 65, wherein the CDP comprises P450.
Claim 67. The method of claim 62 to claim 66, wherein the CDP comprises P450-ferredoxin reductase-ferredoxin fusion protein (P450-FdxR-Fdx).
Claim 68. The method of claim 62 to claim 67, wherein the StAR protein and the CDP comprise at least one mitochondrial signaling protein.
Claim 69. The method of claim 62 to claim 68, wherein the StAR protein and CDP are expressed from a cell-specific promoter sequence.
Claim 70. The method of claim 62 to claim 76, for use in reducing cholesterol in a subject having high cholesterol.
Claim 71. The method of claim 62 to claim 71, wherein at least about 10% of the total amount of StAR protein and CDP in the cell is located in a mitochondrial membrane.
Claim 72. The method of claim 62 to claim 72, wherein the StAR protein or the CDP is expressed from a gene integrated into the mammalian cell's genome.
Claim 73. The method of claim 62 to claim 72, wherein the StAR protein or the CDP is from an extra genomic gene.
Claim 74. The method of claim 62 to claim 73, wherein the patient has a total cholesterol of greater than 200 mg/dl.
Claim 75. The method of claim 62 to claim 74, wherein the patient suffers from non-alcoholic fatty liver disease.
Claim 76. The method of claim 62 to claim 74, wherein the patient suffers from non-alcoholic steatohepatitis.
Claim 77. The method of claim 62 to claim 76, wherein the modified cell is derived from a liver cell, neuronal cell, bone cell, muscle cell, endothelial cell, epithelial cell, immune cell, hematopoietic cell, and stem cell or precursor cell thereof.
Claim 78. The method of claim 62 to claim 77, wherein the method reduces the total cholesterol concentration in the patient's blood by greater than 5%.
Claim 79. A method of modifying a mammalian mitochondrion comprising:
inserting at least one non-native engineered Steroidogenic Acute Regulatory (StAR) protein or portion thereof into the mammalian mitochondrion outer membrane; and
wherein the mitochondrion comprises one or more of a cholesterol degrading protein (CDP) comprising cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).
Claim 80. The method of claim 79, wherein the StAR protein comprises residues 63 to 188 of SEQ ID NO:7.
Claim 81. The method of claim 79 or claim 80, wherein the StAR protein comprises a duplication of residues 63-188 of SEQ ID NO:7.
Claim 82. The method of any of claim 79 to claim 81, wherein the StAR protein comprises at least a portion of the TOMM20 protein.
Claim 83. The method of any of claim 79 to claim 82, wherein the CDP comprises P450.
Claim 84. The method of any of claim 79 to claim 83, wherein the CDP comprises P450-ferredoxin reductase-ferredoxin fusion protein (P450-FdxR-Fdx).
Claim 85. The method of any of claim 79 to claim 84, wherein the StAR protein and the CDP comprise at least one mitochondrial signaling protein.
Claim 86. The method of any of claim 79 to claim 85, wherein the StAR protein and CDP are expressed from a cell-specific promoter sequence.
Claim 87. The method of any of claim 79 to claim 86, for use in treating a subject suffering from or at risk of developing high cholesterol.
Claim 88. The method of any of claim 79 to claim 87, for use in reducing cholesterol in a subject having high cholesterol.
Claim 89. The method of any of claim 79 to claim 88, wherein at least about 10% of the total amount of StAR protein and CDP in the cell is located in a mitochondrial membrane.
Claim 90. The method of any of claim 79 to claim 90, wherein the StAR protein or the CDP is expressed from a gene integrated into the mammalian cell's genome.
Claim 91. The method of any of claim 79 to claim 89, wherein the StAR protein or the CDP is from an extra genomic gene.
Claim 92. The method of any of claim 79 to claim 91, for use in treating a subject suffering from or at risk of developing high cholesterol.
Claim 93. The method of any of claim 79 to claim 92, for use in reducing cholesterol in a subject.
Claim 94. The method of any of claim 79 to claim 93, wherein the cell is selected from a liver cell, neuronal cell, bone cell, muscle cell, endothelial cell, epithelial cell, immune cell, hematopoietic cell, and stem cell or precursor cell thereof.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1. A nucleic acid composition comprising:
a coding sequence for Steroidogenic Acute Regulatory (StAR) protein comprising at least one copy of residues 63 to 188 of SEQ ID NO:7; and
a coding sequence for a cholesterol degrading protein (CDP) comprising one or more of cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx).

2-3. (canceled)

4. The nucleic acid of claim 1, wherein the StAR protein comprises at least a portion of the TOMM20 protein.

5. (canceled)

6. The nucleic acid of claim 1, wherein the CDP comprises P450-ferredoxin reductase-ferredoxin fusion protein (P450-FdxR-Fdx).

7. The nucleic acid of claim 1, wherein the StAR protein and the CDP comprise at least one mitochondrial signaling protein.

8. The nucleic acid of claim 1, wherein the nucleic acid further includes a cell-specific promoter sequence.

9. The nucleic acid of claim 1, wherein the nucleic acid is comprised in a lipo nanoparticle or a viral vector.

10-11.

12. The nucleic acid of claim 1, for use in transforming a mammalian cell in vitro or in vivo.

13-16. (canceled)

17. The nucleic acid of claim 12, for use in reducing cholesterol in a subject having high cholesterol.

18-30. (canceled)

31. A mammalian cell comprising:
an engineered Steroidogenic Acute Regulatory (StAR) protein or portion thereof comprising at least one copy of residues 63 to 188 of SEQ ID NO:7; and
a cholesterol degrading protein (CDP) comprising P450-ferredoxin reductase-ferredoxin fusion protein (P450-FdxR-Fdx).

32-33. (canceled)

34. The mammalian cell of claim 31, wherein the StAR protein comprises at least a portion of the TOMM20 protein.

35-36. (canceled)

37. The mammalian cell of claim 31, wherein the StAR protein and the CDP comprise at least one mitochondrial signaling protein.

38. (canceled)

39. The mammalian cell of claim 31, wherein the StAR protein or the CDP is coded for by a gene integrated into the genome of the mammalian cell or by an extra genomic gene.

40. (canceled)

41. The mammalian cell of claim 39, for use in treating a subject suffering from or at risk of developing high cholesterol.

42. (canceled)

43. The mammalian cell of claim 31, wherein the cell is selected from a liver cell, neuronal cell, bone cell, muscle cell, endothelial cell, epithelial cell, immune cell, hematopoietic cell, and stem cell or precursor cell thereof.

44-61. (canceled)

62. A method of treating a patient at risk of developing or suffering from high cholesterol comprising:
administering to the patient at least one modified cell, the modified cell comprising
a non-native engineered Steroidogenic Acute Regulatory (StAR) protein or portion thereof comprising at least one copy of residues 63 to 188 of SEQ ID NO:7; and
a cholesterol degrading protein (CDP) comprising P450-ferredoxin reductase-ferredoxin fusion protein (P450-FdxR-Fdx).

63-64. (canceled)

65. The method of claim 62, wherein the StAR protein comprises at least a portion of the TOMM20 protein.

66-67. (canceled)

68. The method of claim 62, wherein the StAR protein and the CDP comprise at least one mitochondrial signaling protein.

69. (canceled)

70. The method of claim 62, for use in reducing cholesterol in a subject having high cholesterol, non-alcoholic fatty liver disease or non-alcoholic steatohepatitis.

71. (canceled)

72. The method of claim 62, wherein the StAR protein or the CDP is expressed from a gene integrated into the mammalian cell's genome or from an extra genomic gene.

73-76. (canceled)

77. The method of claim 62, wherein the modified cell is derived from a liver cell, neuronal cell, bone cell, muscle cell, endothelial cell, epithelial cell, immune cell, hematopoietic cell, and stem cell or precursor cell thereof.

78-94. (canceled)
